(19) United States
(12) Patent Application Publication
(10) Pub. No.: US 2025/0072788 A1
(43) Pub. Date: Mar. 6, 2025

(54) GAIT MONITORING AND HEALTHCARE SYSTEM

(71) Applicant: National Tsing Hua University, Hsinchu City (TW)

(72) Inventors: Hung-Yin TSAI, Hsinchu City (TW); Meng-Hsuan TIEN, Hsinchu City (TW); Shaw-Ruey LYU, Chiayi City (TW); U San NG, Hsinchu City (TW); Yi-Hsuan CHEN, Chiayi City (TW)

(21) Appl. No.: 18/489,344

(22) Filed: Oct. 18, 2023

(30) Foreign Application Priority Data
Aug. 29, 2023 (TW) 112132653

(51) Int. Cl. A61B 5/11; A61B 5/00

(52) U.S. Cl. CPC A61B 5/112; A61B 5/0024; A61B 5/4585; A61B 5/6828; A61B 5/7282; A61B 5/746; A61B 2562/0219

(57) ABSTRACT

A gait monitoring and healthcare system includes a first IMU, a second IMU and a local host. The first IMU senses an activity state of a thigh and generates a first activity signal. The second IMU senses an activity state of a calf and generates a second activity signal. The local host includes a microprocessor and an alarm. The microprocessor performs operations of: calculating a first pitch angle between a horizontal plane and the first IMU, and a second pitch angle between the horizontal plane and the second IMU according to the first and second activity signals; calculating activity information of a knee joint, including a bending angle, according to the first and second pitch angles; and judging whether the bending angle of the knee joint falls within a dangerous range according to the activity information, and controlling the alarm to output a warning signal if yes.

### CROSS-REFERENCE TO RELATED APPLICATIONS

This application claims priority of No. 112132653 filed in Taiwan R.O.C. on Aug. 29, 2023 under 35 USC 119, the entire content of which is hereby incorporated by reference.

### FIELD OF THE INVENTION

This disclosure relates to a gait monitoring and healthcare system, and more particularly to a gait or knee-joint activity monitoring and healthcare system for calculating and monitoring a bending angle, an angular velocity and/or a duration of a knee joint according to information of pitch angles.

### DESCRIPTION OF RELATED ART

The knee osteoarthritis has the prevalence of about 15% in Taiwan, there are about 3.5 million people troubled thereby, and there is a tendency that the people becomes younger. The knee osteoarthritis was originally thought to be untreatable, and the joint replacement needs to be performed when the knee osteoarthritis becomes serious. However, there are 75% of people unwilling to have the knee replaced, and the troubles are present. Although there are monitoring systems or devices for the knee joint care on the market, many usage restrictions are present, or even the measured data is not the complete knee joint data.

Taiwan Patent No. TWI615129B discloses a gait analyzing system including a sole sensing unit, a knee sensing unit and a portable device. The portable device performs a gait analysis according to information of the sole sensing unit and the knee sensing unit. However, TWI615129B fails to disclose details of calculations. In addition, the user has the bad feeling of the pressure sensor disposed on the user's sole upon walking, the cost and calculation loading are increased, and the microprocessor of the high-cost portable device is required. In addition, the wireless information transmission may cause delays, so that the real-time monitoring and warning cannot be obtained. Therefore, the prior art needs to further improved.

### SUMMARY OF THE INVENTION

It is therefore an objective of this disclosure to provide a gait monitoring and healthcare system for calculating and monitoring a bending angle, an angular velocity and/or a duration of a knee joint according to pitch angle information, wherein only two inertial measurement units (IMUs) are required to perform sensing.

To achieve the above-identified objective, this disclosure provides a gait monitoring and healthcare system including a first IMU, a second IMU and a local host. The first IMU to be mounted on a thigh of a user senses an activity state of the thigh and generates a first activity signal. The second IMU to be mounted on a calf of the user senses an activity state of the calf and generates a second activity signal. The local host electrically connected to the first IMU and the second IMU includes a microprocessor and an alarm. The microprocessor performs operations of: (a) calculating a first pitch angle between the first IMU and a horizontal plane, and a second pitch angle between the second IMU and the horizontal plane according to the first activity signal and the second activity signal; (b) calculating activity information of a knee joint of the user according to the first pitch angle and the second pitch angle, wherein the activity information includes a bending angle, an angular velocity and a duration of the knee joint; and (c) judging whether the bending angle of the knee joint falls within a dangerous range or not according to the activity information, and controlling the alarm to output a warning signal if yes.

With the above-mentioned embodiment, only two IMUs need to be mounted on the thigh and the calf, so that the pitch angles and the knee joint angle can be calculated and the associated data analysis and warning can be performed.

In order to make the above-mentioned content of this disclosure more obvious and be easily understood, preferred embodiments will be described in detail as follows in conjunction with the accompanying drawings.

### BRIEF DESCRIPTION OF DRAWINGS

FIG. 1 is a block diagram showing a gait monitoring and healthcare system according to a preferred embodiment of this disclosure.

FIG. 2 shows a used state of the gait monitoring and healthcare system of FIG. 1.

FIG. 3 is a simplified schematic view showing the gait monitoring and healthcare system of FIG. 2.

FIG. 4 is a flow chart showing operations of the gait monitoring and healthcare system of FIG. 1.

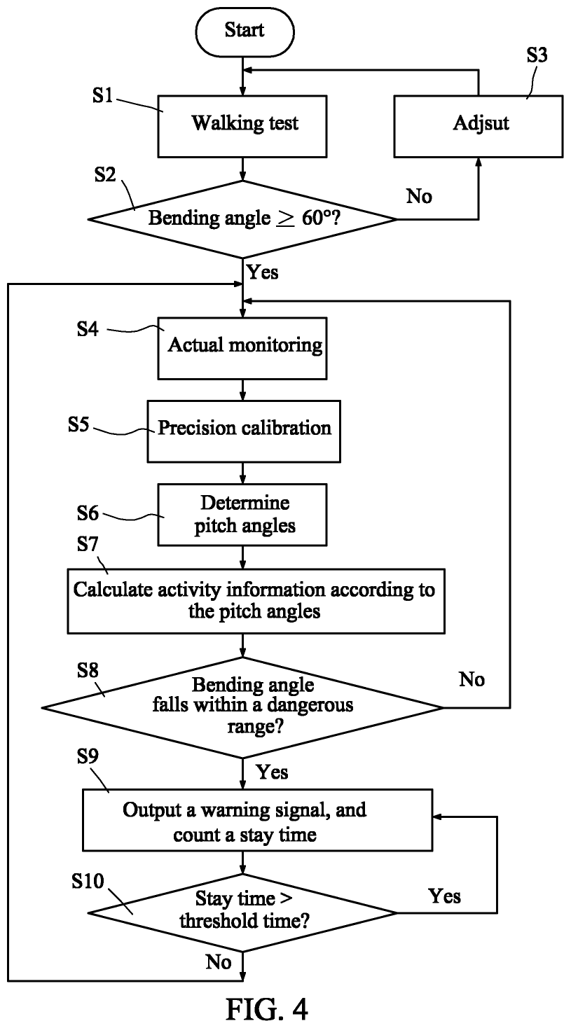

### DETAILED DESCRIPTION OF THE INVENTION

After monitoring, analyzing and researching real activity data of the knee joint, applicants found that the knee osteoarthritis can be improved or even cured by properly using the knee joint. As long as the knee joint of the normal person is properly used, the possibility of suffering from the knee osteoarthritis can be significantly decreased. So, a system and an aid for preventing and treating the knee osteoarthritis is provided to detect and monitor the movement of the knee joint, and provide the correction information according to the bad activity to prevent and improve the knee osteoarthritis.

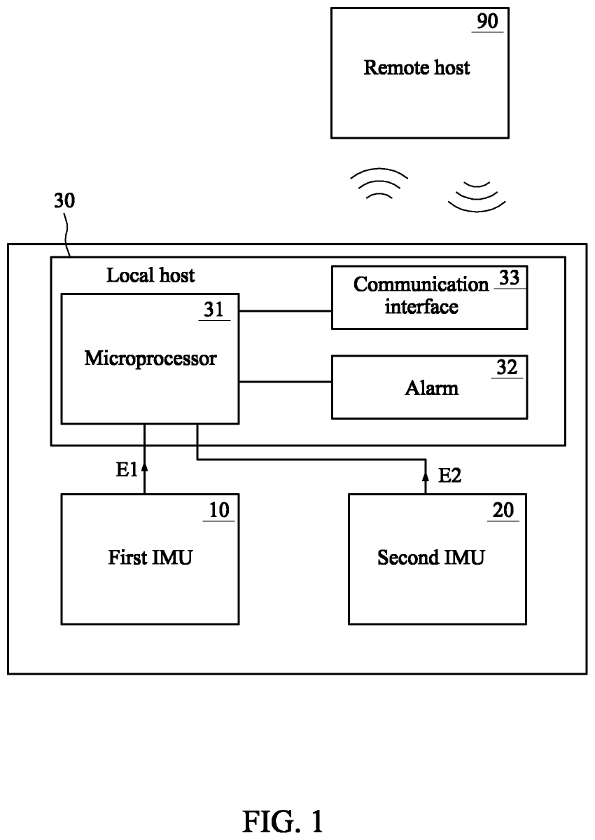

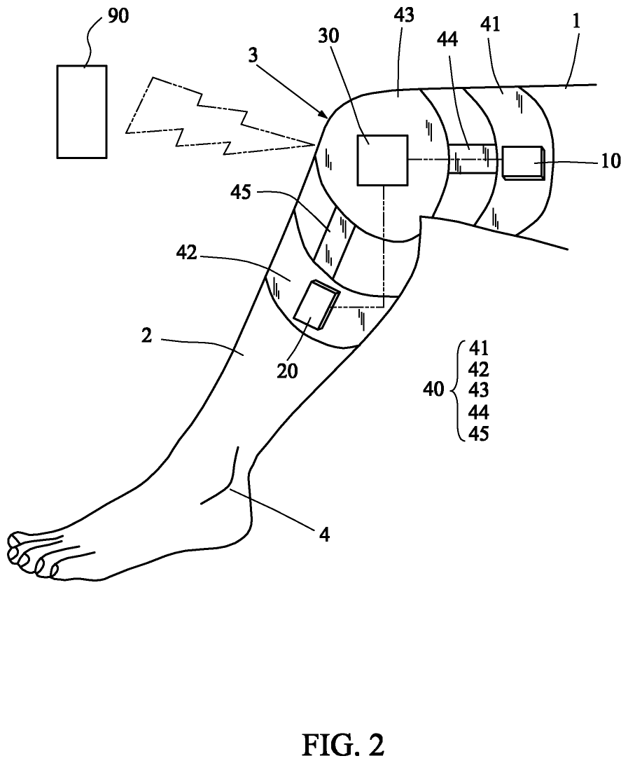

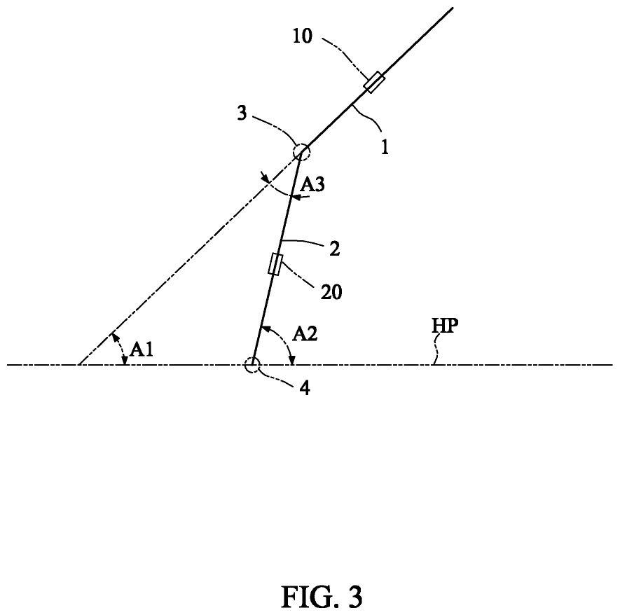

Referring to FIGS. 1 to 3, a gait monitoring and healthcare system 100 of this embodiment includes a first IMU 10, a second IMU 20 and a local host 30. The first IMU 10 to be mounted on a thigh 1 of a user senses an activity state of the thigh 1 and generates a first activity signal E1. The second IMU 20 to be mounted on a calf 2 of the user senses an activity state of the calf 2 and generates a second activity signal E2. The local host 30 is electrically connected to the first IMU 10 and the second IMU 20, and includes a microprocessor 31 and an alarm 32. The microprocessor 31 performs the following operations.

First, a first pitch angle A1 between a horizontal plane HP and the first IMU 10 (or thigh 1), and a second pitch angle A2 between the horizontal plane HP and the second IMU 20 (or calf 2) are calculated according to the first activity signal E1 and the second activity signal E2. The gait judgement can be advantageously made according to the pitch angles. For example, the user's thigh is lifted higher upon walking upstairs and downstairs, and the knee joint movements are similar when the user walks uphill, downhill and horizontally. However, the movement ranges of the independent thigh and calf are not the same, and the judgement cannot be made according to the knee joint angle. Thus, the gait judgement can be simplified with the aid of the moving angle (first pitch angle) of the thigh and the moving angle (second pitch angle) of the calf.

Next, activity information of a knee joint 3 of the user is calculated according to the first pitch angle A1 and the second pitch angle A2. The activity information includes a bending angle (or knee joint angle) A3, an angular velocity and a duration of the knee joint 3, where A3=180-A1-A2 degrees.

Then, it is judged whether the bending angle A3 of the knee joint 3 (angular velocity and duration may be optionally added) in an activity state (e.g. walking or running state) falls within a dangerous range according to the activity information. If yes, the microprocessor 31 controls the alarm 32 to output a warning signal. The alarm 32 includes, for example but without limitation to, a buzzer, a vibrator and the like. The activity state data can be calculated and the user's current activity can be predicted to achieve the knee-joint activity monitoring and healthcare function according to the above-mentioned elements. Herein, the local host 30 is disposed on the knee joint 3, so that the knee joint 3 can directly feel the vibration, and the better reminder effect can be achieved. The calculation based on the pitch angles can provide the useful data for the microprocessor 31. For example, when the user moves on a horizontal ground, the working angle of an ankle joint 4 can be provided, so that the microprocessor 31 obtains other judging bases, and the microprocessor 31 can conveniently calculate the first pitch angle A1 and the second pitch angle A2 using a quaternion method. The IMU includes a 3-axis gyroscope. Initial values (e.g., (1, 0, 0, 0)) of the real numbers (90, 91, 92, 93) of the quaternion q are set according to (Equation 1), and the quaternion is updated into q′ using the angular velocity variation of the gyroscope according to (Equation 2), q′ is normalized into a new q, and a pitch angle PA is determined according to (Equation 3).

$$q = {q}_{0} + {q}_{1} i + {q}_{2} j + {q}_{3} k \qquad ( Equation 1 )$$

$${q}_{0}^{′} = {q}_{0} + 0.5 \times ( - {q}_{1} \times {g}_{x} - {q}_{2} \times {g}_{y} - {q}_{3} \times {g}_{z} ) \times Δ T ; \qquad ( Equation 2 )$$

$${q}_{1}^{′} = {q}_{1} + 0.5 \times ( {q}_{0} \times {g}_{x} + {q}_{2} \times {g}_{z} - {q}_{3} \times {g}_{y} ) \times Δ T ;$$

$${q}_{2}^{′} = {q}_{2} + 0.5 \times ( {q}_{0} \times {g}_{y} - {q}_{1} \times {g}_{z} + {q}_{3} \times {g}_{x} ) \times Δ T ;$$

$${q}_{3}^{′} = {q}_{3} + 0.5 \times ( {q}_{0} \times {g}_{z} + {q}_{1} \times {g}_{y} - {q}_{2} \times {g}_{x} ) \times Δ T ;$$

$$PA = {sin}^{- 1} ( 2 ( {q}_{0} \times {q}_{2} - {q}_{3} \times {q}_{1} ) ) \qquad ( Equation 3 )$$

where i, j, k are imaginary numbers, and ΔT is a sampling update time of the IMU.

In one example, the dangerous range ranges from 60 to 120 degrees, and the bending angle A3 is the included angle between an extension line of the thigh 1 and the calf 2. The applicants have found that a destructive pressure against the knee's cartilage is generated when the bending angle A3 ranges from 60 to 120 degrees in the walking state, and the pressure sensor on the sole is not needed. The consideration of the duration is because that the damage gets much more as the damage pressure is kept longer. Thus, if the bending angle A3 falls within the dangerous range, then the microprocessor 31 further counts the duration of the bending angle A3 to obtain a stay time. If the stay time is longer than a threshold time, then the microprocessor 31 further controls the alarm 32 to output another warning signal to prevent the knee joint from being damaged. In order to satisfy different conditions, the microprocessor 31 determines the threshold time according to the user's age (previously inputted to the local host 30), the activity information and the activity state (e.g., the medium, high and low speed walking state or running state). It is understandable that the microprocessor 31 may also perform warning according to the dangerous ranges of other activities or stationary bending angles of the knee joints and the threshold time values.

In this example, a nine-axis IMU is used. The IMU contains a 3-axis gyroscope, a 3-axis accelerometer and a 3-axis magnetometer for respectively measuring values of the angular velocity, acceleration and magnetic field strength of an object in a three-dimensional space to obtain sensing values. The microprocessor 31 performs a knee-joint activity analysis according to these sensing values. In order to increase the precision, the microprocessor 31 calibrates the sensing values of the gyroscope according to the sensing values of one or two of the accelerometer and the magnetometer, then the first pitch angle and the second pitch angle are calculated using the quaternion method, and then calculates activity information of the bending angle, angular velocity, duration and altitude variation of the knee joint and the like according to the first pitch angle and the second pitch angle.

For example, the Mahony algorithm is used. The initial values (1, 0,0,0) of (q₀, q₁, q₂, q₃) are set according to (Equation 1). Then, values (gₓ, g_{y}, g_{z}) of the gyroscope are calibrated according to values (aₓ, a_{y}, a_{z}) of the accelerometer or values (mₓ, m_{y}, m_{z}) of the magnetometer, and (q₀, q₁, q₂, q₃) are updated. In the example of performing the calibration using the values of the accelerometer, the theoretical gravitational acceleration vector (vₓ, v_{y}, v_{z}) is determined according to values (q₀, q₁, q₂, q₃) of q and (Equation 4).

$${v}_{x} = {q}_{1} \times {q}_{3} - {q}_{0} \times {q}_{2} ; \qquad ( Equation 4 )$$

$${v}_{y} = {q}_{0} \times {q}_{1} + {q}_{2} \times {q}_{3} ;$$

$${v}_{z} = {q}_{0} \times {q}_{0} + {q}_{3} \times {q}_{3} - 0.5 ;$$

Then, (aₓ, a_{y}, a_{z}) are normalized into (bₓ, b_{y}, b_{z}) according to (Equation 5), and errors (eₓ, e_{y}, e_{z}) between the actual gravitational acceleration and theoretical acceleration are determined according to (Equation 6).

$${b}_{x} = {a}_{x} / \sqrt{{a}_{x}^{2} + {a}_{y}^{2} + {a}_{z}^{2}} ; \qquad ( Equation 5 )$$

$${b}_{y} = {a}_{y} / \sqrt{{a}_{x}^{2} + {a}_{y}^{2} + {a}_{z}^{2}} ;$$

$${b}_{z} = {a}_{z} / \sqrt{{a}_{x}^{2} + {a}_{y}^{2} + {a}_{z}^{2}} ;$$

$${e}_{x} = {b}_{y} \times {v}_{z} - {b}_{z} \times {v}_{y} ; \qquad ( Equation 6 )$$

$${e}_{y} = {b}_{z} \times {v}_{x} - {b}_{x} \times {v}_{z} ;$$

$${e}_{z} = {b}_{x} \times {v}_{y} - {b}_{y} \times {v}_{x} ;$$

Next, because integration is required, a parameter item i=iₓ+i_{y}+i_{z} is set to have the initial value i=0, +0, +02, and (gₓ, g_{y}, g_{z}) is calibrated according to coefficients ki, kp and ΔT to obtain calibrated gyroscope values (gₓ′, g_{y}′, g_{z}′) according to (Equation 7).

$${i}_{x}^{′} = {i}_{x} + ki \times {e}_{x} \times Δ T ; \qquad ( Equation 7 )$$

$${i}_{y}^{′} = {i}_{y} + ki \times {e}_{y} \times Δ T ;$$

$${i}_{z}^{′} = {i}_{z} + ki \times {e}_{z} \times Δ T ;$$

$${g}_{x}^{′} = {g}_{x} + {i}_{x}^{′} + kp \times {e}_{x} ;$$

$${g}_{y}^{′} = {g}_{y} + {i}_{y}^{′} + kp \times {e}_{y} ;$$

$${g}_{z}^{′} = {g}_{z} + {i}_{z}^{′} + kp \times {e}_{z} ;$$

In one example, ki=30; and kp=0 are taken.

Then, (q₀, q₁, q₂, q₃) are updated into the real numbers (q₀′, q₁′, q₂′, q₃′) of the final quaternion according to (Equation 8).

$${g}_{x}^{′′} = {g}_{x}^{′} \times 0.5 \times Δ T ; \qquad ( Equation 8 )$$

$${g}_{y}^{′′} = {g}_{y}^{′} \times 0.5 \times Δ T ;$$

$${g}_{z}^{′′} = {g}_{z}^{′} \times 0.5 \times Δ T ;$$

$${q}_{0}^{′} = {q}_{0} + ( - {q}_{1} \times {g}_{x}^{′′} - {q}_{2} \times {g}_{y}^{′′} - {q}_{3} \times {g}_{z}^{′′} ) ;$$

$${q}_{1}^{′} = {q}_{1} + ( {q}_{0} \times {g}_{x}^{′′} + {q}_{2} \times {g}_{z}^{′′} - {q}_{3} \times {g}_{y}^{′′} ) ;$$

$${q}_{2}^{′} = {q}_{2} + ( {q}_{0} \times {g}_{y}^{′′} - {q}_{1} \times {g}_{z}^{′′} + {q}_{3} \times {g}_{x}^{′′} ) ;$$

$${q}_{3}^{′} = {q}_{3} + ( {q}_{0} \times {g}_{z}^{′′} + {q}_{1} \times {g}_{y}^{′′} - {q}_{2} \times {g}_{x}^{′′} ) ;$$

Finally, (q₀′, q₁′, q₂′, q₃′) are normalized into new (q₀, q₁, q₂, q₃) according to (Equation 9), and the pitch angle is obtained according to (Equation 3). It is understandable, the calibration method using the magnetometer is similar to that using the accelerometer, and detailed descriptions thereof will be omitted. The advantage is that the pitch angles can be directly obtained using the quaternion. It is unnecessary to convert the sensing data of the IMU into the rotation coordinate of the IMU, and then to determine the included angle between the two IMUs, so that the calculation becomes convenient.

$${q}_{0} = {q}_{0}^{′} / \sqrt{{q}_{0}^{′2} + {q}_{1}^{′2} + {q}_{2}^{′2} + {q}_{3}^{′2}} ; \qquad ( Equation 9 )$$

$${q}_{1} = {q}_{1}^{′} / \sqrt{{q}_{0}^{′2} + {q}_{1}^{′2} + {q}_{2}^{′2} + {q}_{3}^{′2}} ;$$

$${q}_{2} = {q}_{2}^{′} / \sqrt{{q}_{0}^{′2} + {q}_{1}^{′2} + {q}_{2}^{′2} + {q}_{3}^{′2}} ;$$

$${q}_{3} = {q}_{3}^{′} / \sqrt{{q}_{0}^{′2} + {q}_{1}^{′2} + {q}_{2}^{′2} + {q}_{3}^{′2}} ;$$

In addition, the activity information is inputted to a machine learning model in the microprocessor 31, which classifies the walking state of the user including the state of walking upstairs, the state of walking downstairs, the state of walking uphill, the state of walking downhill and the state of walking horizontally. The machine learning model may be a support vector machine (SVM), a multi-layer perceptron (MLP) and the like.

The gait monitoring and healthcare system 100 may further include an aid 40. The first IMU 10 is disposed on the thigh 1 through a first mounting part 41 of the aid 40. The second IMU 20 is disposed on the calf 2 through a second mounting part 42 of the aid 40. The aid 40 may be a wearable having a fitting part 43 for accommodating the knee joint 3. A first distance from a center point of the fitting part 43 to the first IMU 10, and a second distance from the center point of the fitting part 43 to the second IMU 20 are greater than or equal to 15 cm. This can solve the calculation error upon actual sensing. The user can wear the aid and then perform tests. Alternatively, the aid 40 may further have a first connection part 44 disposed between and connected to the first mounting part 41 and the fitting part 43, and a second connection part 45 disposed between and connected to the second mounting part 42 and the fitting part 43 so that the user may let the first mounting part 41 and the second connection part 45 in flat surface contact with skin surfaces of the thigh and the calf, and the above-mentioned condition can be satisfied.

The gait monitoring and healthcare system 100 may further include a remote host 90 communicating with or connected to a communication interface 33 of the local host 30. The remote host may be a mobile phone, a tablet computer, a notebook computer or a server. The microprocessor 31 outputs the activity information of the knee joint to the remote host 90 through the communication interface 33. The remote host 90 performs a statistical analysis on the activity information, and provides an activity correction solution to the user according to the activity information, so that the information of correcting the user's bad activity can be provided. Thus, this system can perform the historical integration and analysis on the user's gait information to obtain the error count, the stay time of the dangerous angle and the like everyday, and further provide the appropriate activity correction solution. The user may also decide some parameters of the correction solution, and trace the progress curve. In addition, personalized gait improvement programs based on factors, such as ages, activity state and the like affecting the knee joints, can be made according to different ethnic groups, the personalized gait or activity suggestions can be customized, and the lifetime of the knee joint can be lengthened.

In summary, this disclosure also provides a gait monitoring and healthcare method including steps S1 to S10.

After the user wears the aid, a walking test is performed (S1). The applicants have found that the calculated knee joint angle is smaller than 60 degrees when the first distance and the second distance are smaller than 15 cm, so that the subsequent errors are generated. Therefore, it is necessary to judge whether the bending angle (knee joint angle) is greater than or equal to 60 degrees (S2). When the knee joint angle is smaller than 60 degrees, the system reminds the user to adjust (S3). When the knee joint angle is greater than or equal to 60 degrees, the actual monitor is performed (S4), the precision calibration is optionally performed (S5), the pitch angles (S6) are determined, and the activity information is calculated (S7). Next, it is judged whether the knee joint angle falls within the dangerous range (S8). If not, the process returns to the step S4. If yes, then the warning signal is outputted, and the stay time is counted (S9). Next, it is judged whether the stay time is longer than the threshold time (S10). If not, the process returns to the step S4. If yes, then the process returns to the step S9.

Compared with the prior art, which needs the additional pressure judgement on the sole or knee, this disclosure only needs two IMUs mounted on the calf and the thigh to make the data analysis and warning possible. In addition, this system can provide the corresponding warning or suggest the activity manner according to the gait analysis result and the personal physiological information (e.g., age), and the user can also configure the personalized training contents and trace the progress curve using this system.

The specific embodiments proposed in the detailed description of this disclosure are only used to facilitate the description of the technical contents of this disclosure, and do not narrowly limit this disclosure to the above-mentioned embodiments. Various changes of implementations made without departing from the spirit of this disclosure and the scope of the claims are deemed as falling within the following claims.

1. A gait monitoring and healthcare system, comprising:
a first IMU to be mounted on a thigh of a user, wherein the first IMU senses an activity state of the thigh and generates a first activity signal;
a second IMU to be mounted on a calf of the user, wherein the second IMU senses an activity state of the calf and generates a second activity signal; and
a local host electrically connected to the first IMU and the second IMU, wherein the local host comprises a microprocessor and an alarm, and performs operations of:
(a) calculating a first pitch angle between the first IMU and a horizontal plane, and a second pitch angle between the second IMU and the horizontal plane according to the first activity signal and the second activity signal;
(b) calculating activity information of a knee joint of the user according to the first pitch angle and the second pitch angle, wherein the activity information comprises a bending angle, an angular velocity and a duration of the knee joint; and
(c) judging whether the bending angle of the knee joint in a walking state falls within a dangerous range or not according to the activity information, and controlling the alarm to output a warning signal if yes.

2. The gait monitoring and healthcare system according to claim 1, wherein the microprocessor performs the operation (a) using a quaternion method.

3. The gait monitoring and healthcare system according to claim 1, wherein the dangerous range ranges from 60 to 120 degrees, and the bending angle is an included angle between an extension line of the thigh and the calf.

4. The gait monitoring and healthcare system according to claim 1, further comprising an aid, wherein the first IMU is disposed on the thigh through a first mounting part of the aid, the second IMU is disposed on the calf through a second mounting part of the aid, a fitting part of the aid accommodates the knee joint, and a first distance from a center point of the fitting part to the first IMU, and a second distance from the center point of the fitting part to the second IMU are greater than or equal to 15 cm.

5. The gait monitoring and healthcare system according to claim 4, wherein the local host is mounted on the knee joint through the fitting part, and the aid further has a first connection part between the first mounting part and the fitting part, and a second connection part between the second mounting part and the fitting part.

6. The gait monitoring and healthcare system according to claim 1, wherein if the bending angle falls within the dangerous range, then the microprocessor further counts the duration of the bending angle to obtain a stay time, wherein if the stay time is longer than a threshold time, then the microprocessor further controls the alarm to output another warning signal.

7. The gait monitoring and healthcare system according to claim 6, wherein the microprocessor determines the threshold time according to an age, activity information and an activity state of the user.

8. The gait monitoring and healthcare system according to claim 1, wherein the first IMU and the second IMU are nine-axis IMUs, each of the nine-axis IMUs comprises an accelerometer, a magnetometer and a gyroscope, and the microprocessor calibrates sensing values of the gyroscope according to sensing values of one or two of the accelerometer and the magnetometer, and then performs the operation (a) to increase precision.

9. The gait monitoring and healthcare system according to claim 1, wherein the activity information is inputted to a machine learning model of the microprocessor, which further classifies the walking state of the user, wherein the walking state comprises a state of walking upstairs, a state of walking downstairs, a state of walking uphill, a state of walking downhill, and a state of walking horizontally.

10. The gait monitoring and healthcare system according to claim 1, further comprising a remote host communicating with a communication interface of the local host, wherein the microprocessor outputs the activity information to the remote host through the communication interface, and the remote host performs a statistical analysis on the activity information, and provides an activity correction solution to the user according to the activity information.
